(19) United States
(12) Patent Application Publication
(10) Pub. No.: US 2002/0161002 A1
(43) Pub. Date: Oct. 31, 2002

(54) Use of catecholamine reuptake inhibitors to enhance memory

(76) Inventors: Mel Epstein, Bristol, RI (US); Kjesten A. Wiig, Providence, RI (US)

Correspondence Address: ROPES & GRAY, ONE INTERNATIONAL PLACE, BOSTON, MA, 02110-2624, US

(21) Appl. No.: 10/039,229

(22) Filed: Jan. 2, 2002

Related U.S. Application Data
(60) Provisional application No. 60/259,374, filed on Jan. 2, 2001.

(51) Int. Cl. A61K 31/551; A61K 31/137

(52) U.S. Cl. 514/220; 514/649

(57) ABSTRACT

The present invention makes available methods and reagents for enhancing memory, e.g., to increase memory function such as long-term memory and recall ability.

### REFERENCE TO RELATED APPLICATIONS

This application claims priority to U.S. Provisional Patent Application No. 60/259,374 filed on Jan. 2, 2001, the specification of which is incorporated by reference herein.

### BACKGROUND OF THE INVENTION

The term “memory” subsumes many different processes and requires the function of many different brain areas. Overall, human memory provides declarative recall, e.g., for facts and events accessible to conscious recollection, and non-declarative recall, e.g., procedural memory of skills and operations not stored regarding time and place. Research in recent years has provided information necessary to many of the various components of memory and identify associated brain regions. A newly acquired experience initially is susceptible to various forms of disruption. With time, however, the new experience becomes resistant to disruption. This observation has been interpreted to indicate that a labile, working, short-term memory is consolidated into a more stable, long-term memory.

Behavioral research has found that the human mind consolidates memory at certain key time intervals. The initial phase of memory consolidation occurs in the first few minutes after we are exposed to a new idea or learning experience. The next phase occurs over a longer period of time, such as during sleep. If a learning experience has on-going meaning to us, the next week or so serves as a further period of memory consolidation. In effect, in this phase, the memory moves from short-term to long-term storage.

Moreover, various mechanisms have been proposed to account for the formation of long-term memory. A wide range of observations suggest an evolutionarily conserved molecular mechanism involved with the formation of long-term memory. These include increased release of synaptic transmitter, increased number of synaptic receptors, decreased Km of receptors, synthesis of new memory factors either in the presynaptic or postsynaptic element, sprouting of new synaptic connections, increase of the active area in the presynaptic membrane and many others. Synaptic plasticity, the change in the strength of neuronal connections in the brain, is thought to underlie long-term memory storage.

“Memory consolidation”, or long-term memory is also believed to play a crucial role in a variety of neurological and mental disorders, including mental retardation, Alzheimer's disease and depression. Indeed, loss or impairment of long-term memory is significant feature of such diseases, and no effective therapy for that effect has emerged. Short-term, or “working” memory, is generally not significantly impaired in such patients.

It is, accordingly, an object of the present invention to provide methods and compositions for enhancing long-term memory function and/or performance. It is also an object of the present invention to provide methods and compositions for prophylactically (e.g., as a neuroprotective treatment) preventing or slowing degradation of long-term memory function and/or performance. It is also an object of the present invention to provide methods and compositions for restoring long-term memory function and/or performance.

### BRIEF SUMMARY OF THE INVENTION

The present invention relates to the discovery that catecholamine reuptake inhibitors can be used to enhance, prevent and/or restore long-term memory function and performance, e.g., to improve long-term memory (LTM) in animal subjects. One aspect of the present invention relates to a method for enhancing memory consolidation in an animal, comprising administering to the animal a formulation of a catecholamine reuptake inhibitor in an amount sufficient to enhance long-term memory in the animal.

In certain preferred embodiments, the catecholamine reuptake inhibitor is a norepinephrine reuptake inhibitor, such as a tertiary amine tricyclic or a secondary amine tricyclic. Exemplary norepinephrine reuptake inhibitors include those selected from the group consisting of amitriptyline, clomipramine, doxepin, imipramine, trimipramine, amoxapine, desipramine, maprotiline, nortriptyline, protriptyline, reboxetine, duloxetine, venlafaxine, milnacipran, mazindol, methylphenidate, nefazodone, nisoxetine, sibutramine and nomifensine.

In certain embodiments, the norepinephrine reuptake inhibitor has one or more of the following characteristics:

a. inhibits presynaptic norepinephrine reuptake with a Ki of 100 nM or less;

b. has over 10 times greater selectivity for blocking norepinephrine reuptake as compared to inhibition of dopamine and serotonin (5-HT) reuptake; and/or

c. is at least 10 times more potent at blocking noradrenergic neurons as compared to serotonergic neurons.

In certain embodiments, the subject method is carried out as part of a therapeutic regimen which includes dosing the animal with a neuronal growth factor, a neuronal survival factor, a neuronal tropic factor, a cholinergic activator, an adrenergic activator, a dopaminergic activator, a glutaminergic activator or an agent that stimulates the PKC or PKA pathways.

In certain embodiments of the kits, preparations, composition and methods, the invention features one or more catecholamine reuptake inhibitors provided in an amount sufficient to enhance long-term memory in a patient by a statistically significant amount when assessed by one or more of a Cambridge Neuropsychological Test Automated Battery (CANTAB); a Children's Memory Scale (CMS); a Contextual Memory Test; a Continuous Recognition Memory Test (CMRT); a Denman Neuropsychology Memory Scale; a Fuld Object Memory Evaluation (FOME); a Graham-Kendall Memory for Designs Test; a Guild Memory Test; a Learning and Memory Battery (LAMB); a Memory Assessment Clinic Self-Rating Scale (MAC-S); a Memory Assessment Scales (MAS); a Randt Memory Test; a Recognition Memory Test (RMT); a Rivermead Behavioral Memory Test; a Russell's Version of the Wechsler Memory Scale (RWMS); a Test of Memory and Learning (TOMAL); a Vermont Memory Scale (VMS); a Wechsler Memory Scale; and a Wide Range Assessment of Memory and Learning (WRAML).

In certain embodiments of the kits, preparations, compositions and methods, the invention features one or more catecholamine reuptake inhibitors provided in an amount sufficient to enhance long-term memory in a patient by a statistically significant amount when assessed by a Providence Recognition Memory Test.

The subject kits, preparations, compositions and methods of the invention can be used for treating and/or preventing memory impairment, wherein the memory impairment results from one or more of anxiety, depression, age-associated memory impairment, minimal cognitive impairment, amnesia, dementia, learning disabilities, memory impairment associated with toxicant exposure, brain injury, brain aneurysm, Parkinson's disease, head trauma, Huntington's disease, Pick's disease, Creutzfeldt-Jakob disease, stroke, schizophrenia, epilepsy, mental retardation, Alzheimer's disease, age, attention deficit disorder, attention deficit hyperactivity disorder, or AIDS-related dementia.

In certain embodiments, the subject method is used for veterinary treatment of a non-human mammal. In other embodiments, the subject method is used for treatment of a human patient.

Another aspect of the invention provides a medicament for enhancing memory consolidation in an animal, comprising a formulation of a catecholamine reuptake inhibitor in an amount sufficient to enhance long-term memory in the animal.

Still another aspect of the invention relates to a method for preparing a formulation for enhancing memory consolidation in an animal, comprising preparing a pharmaceutical preparation comprising one or more catecholamine reuptake inhibitors in an amount sufficient to enhance long-term memory in the animal.

In certain embodiments, the invention features a kit comprising an catecholamine reuptake inhibitor(s), e.g., as described herein and preferably provided in single oral dosage form or as a transdermal patch for enhancing memory in a patient (preferably a human), and in association with instructions (written and/or pictorial) describing the use of the formulation for enhancing memory, and optionally, warnings of possible side effects and drug-drug or drug-food interactions.

Another aspect of the invention relates to a method for conducting a pharmaceutical business, which includes: (a) manufacturing the kits, preparations, and compositions of the present invention; and (b) marketing to healthcare providers the benefits of using the kits, preparations, and compositions of the present invention to enhance memory of treated patients.

Another aspect of the invention relates to a method for conducting a pharmaceutical business, comprising: (a) providing a distribution network for selling the kits, preparations, and compositions of the present invention; and (b) providing instruction material to patients or physicians for using the kits, preparations, and compositions of the present invention to enhance memory of treated patients.

Yet another aspect of the invention relates to a method for conducting a pharmaceutical business, comprising: (a) determining an appropriate dosage of a catecholamine reuptake inhibitor(s0 to enhance memory function in a class of patients; (b) conducting therapeutic profiling of one or more formulations of the catecholamine reuptake inhibitor(s) identified in step (a), for efficacy and toxicity in animals; and (c) providing a distribution network for selling the formulations identified in step (b) as having an acceptable therapeutic profile.

For instance, the subject business method can include an additional step of providing a sales group for marketing the preparation to healthcare providers.

Another aspect of the invention relates to a method for conducting a pharmaceutical business, comprising: (a) determining an appropriate dosage of a catecholamine reuptake inhibitor(s) to enhance memory function in a class of patients; and (b) licensing, to a third party, the rights for further development and sale of the catecholamine reuptake inhibitor(s) for enhancing memory.

In certain embodiments of the method, the class of patients suffer from memory impairment. In preferred embodiments of the method, the memory impairment results from one or more of anxiety, depression, age-associated memory impairment, minimal cognitive impairment, amnesia, dementia, learning disabilities, memory impairment associated with toxicant exposure, brain injury, brain aneurysm, Parkinson's disease, head trauma, Huntington's disease, Pick's disease, Creutzfeldt-Jakob disease, stroke, schizophrenia, epilepsy, mental retardation, Alzheimer's disease, age, attention deficit disorder, attention deficit hyperactivity disorder, or AIDS-related dementia. In other preferred embodiments of the method, the class of patients are normal individuals.

### BRIEF DESCRIPTION OF THE DRAWINGS

FIG. 1 presents the effectiveness of various doses of methylphenidate on latency in passive avoidance testing, an indicator of memory consolidation.

FIG. 2 demonstrates the effect of methylphenidate on latency in passive avoidance testing.

FIG. 3 depicts the effects of methylphenidate on normal and fornix-lesioned animals.

FIG. 4 presents the effectiveness of various doses of amphetamine.

FIG. 5 demonstrates the effect of amphetamine on memory retention.

FIG. 6 shows the varying effect of amphetamine depending on the time between administration and inception of training.

FIG. 7 illustrates the effect of amphetamine on memory retention one week after the initial training.

FIG. 8 depicts the effects of amphetamine on normal and fornix-lesioned animals.

FIG. 9 shows the effect of S-(+)amphetamine on memory retention.

FIG. 10 shows the effect of S-(+)amphetamine on the Total Distance Activity Level test.

FIG. 11 shows the effect of S-(+)amphetamine on the Total Movement Activity Level test.

FIG. 12 shows the effect of S-(+)amphetamine on the Total Rest Time Activity Level test.

FIG. 13 shows the effect of S-(+)amphetamine on the Amount of Rearing Activity Level test.

FIG. 14 presents the effectiveness of various doses of R-(−)amphetamine on memory retention.

FIG. 15 demonstrates the effectiveness of R-(−)amphetamine on memory retention.

FIG. 16 shows the effect of R-(−)amphetamine on the Total Distance Activity Level test.

FIG. 17 shows the effect of R-(−)amphetamine on the Number of Movements Activity Level test.

FIG. 18 shows the effect of R-(−)amphetamine on the Movement Time Activity Level test.

FIG. 19 shows the effect of R-(−)amphetamine on the Number of Rears Activity Level test.

FIG. 20 shows the effect of R-(−)amphetamine on the Number of Stereotyped Movements Activity Level test.

FIG. 21 shows the effect of R-(−)amphetamine on the Time Spent Resting Activity Level test.

FIG. 22 depicts certain exemplary structures of norepinephrine reuptake inhibitors.

### DETAILED DESCRIPTION OF THE INVENTION

I. Overview

The present invention relates to the discovery that the catecholamine reuptake inhibitors, especially norepinephrine reuptake inhibitors, can be used to enhance and/or restore long-term memory function and performance, e.g., to improve long-term memory (LTM) in animal subjects.

The present invention is based on utilizing a composition which includes one or more catecholamine reuptake inhibitors for increasing long-term potentiation and/or improving long-term memory in animals, such as humans. The formulation includes a therapeutic amount of the catecholamine reuptake inhibitor(s) necessary to affect memory enhancement.

In certain embodiments, the method includes administering, conjointly with the pharmaceutical preparation, one or more of a neuronal growth factor, a neuronal survival factor, and a neuronal tropic factor. Additionally or alternatively, a subject compound may be administered in conjunction with a cholinergic, adrenergic, dopaminergic, or glutaminergic activator. An agent to be administered conjointly with a subject compound may be formulated together with a subject compound as a single pharmaceutical preparation, e.g., as a pill or other medicament including both agents, or may be administered as a separate pharmaceutical preparation.

In another aspect, the present invention provides pharmaceutical preparations comprising, as an active ingredient, methylphenidate or a derivative thereof. The subject reuptake inhibitor is formulated in an amount sufficient to improve LTP in an animal. The subject preparations and methods can be treatments using reuptake inhibitors effective for human and/or animal subjects. In addition to humans, other animal subjects to which the invention is applicable extend to both domestic animals and livestock, raised either as pets or for commercial purposes. Examples are dogs, cats, cattle, horses, sheep, bogs, and goats.

Still another aspect of the invention relates to the use of reuptake inhibitors for lessening the severity or prophylactically preventing the occurrence of learning and/or memory defects in an animal, and thus, altering the learning ability and/or memory capacity of the animal. As a result, the compounds of the present invention may be useful for treating and/or preventing memory impairment, e.g., due to toxicant exposure, brain injury, age-associated memory impairment, mild cognitive impairment, epilepsy, mental retardation in children, and dementia resulting from a disease, such as Parkinson's disease, Alzheimer's disease, AIDS, head trauma, Huntington's disease, Pick's disease, Creutzfeldt-Jakob disease, and stroke. In addition, the compounds of the invention may be useful in enhancing memory in normal individuals.

The invention also relates to the conjoint use of a reuptake inhibitor with agents that mimic or stimulate PKC and/or PKA pathways.

II. Definitions

For convenience, certain terms employed in the specification, examples, and appended claims are collected here.

The term “ED₅₀” means the dose of a drug which produces 50% of its maximum response or effect.

An “effective amount” of, e.g., a reuptake inhibitor, with respect to the subject method of treatment, refers to an amount of the activator in a preparation which, when applied as part of a desired dosage regimen brings about enhanced LTM according to clinically acceptable standards.

The term “LD₅₀” means the dose of a drug which is lethal in 50% of test subjects.

A “patient” or “subject” to be treated by the subject method can mean either a human or non-human animal.

The term “prodrug” is intended to encompass compounds which, under physiologic conditions, are converted into the therapeutically active agents of the present invention. A common method for making a prodrug is to include selected moieties which are hydrolyzed under physiologic conditions to reveal the desired molecule. In other embodiments, the prodrug is converted by an enzymatic activity of the host animal.

The term “metabolites” refers to active derivatives produced upon introduction of a compound into a biological milieu, such as a patient.

The term “therapeutic index” refers to the therapeutic index of a drug defined as LD₅₀/ED₅₀.

By “transdermal patch” is meant a system capable of delivery of a drug to a patient via the skin, or any suitable external surface, including mucosal membranes, such as those found inside the mouth. Such delivery systems generally comprise a flexible backing, an adhesive and a drug retaining matrix, the backing protecting the adhesive and matrix and the adhesive holding the whole on the skin of the patient. On contact with the skin, the drug-retaining matrix delivers drug to the skin, the drug then passing through the skin into the patient's system.

The term “catecholamines” refers to neurotransmitters that have a catechol ring (e.g., a 3,4-dihydroxylated benzene ring). Examples are dopamine, norepinephrine, and epinephrine.

The term “statistically significant” as used herein means that the obtained results are not likely to be due to chance fluctuations at the specified level of probability. The two most commonly specified levels of significance are 0.05 (p=0.05) and 0.01 (p=0.01). The level of significance equal to 0.05 and 0.01 means that the probability of error is 5 out of 100 and 1 out of 100, respectively.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

Contemplated equivalents of the compounds described above include compounds which otherwise correspond thereto, and which have the same general properties thereof (e.g., the ability to effect long-term memory), wherein one or more simple variations of substituents are made which do not adversely affect the efficacy of the compound. In general, the compounds of the present invention may be prepared by the methods described below, or by modifications thereof, using readily available starting materials, reagents and conventional synthesis procedures. In these reactions, it is also possible to make use of variants which are in themselves known, but are not mentioned here.

III. Exemplary Compounds of the Invention.

A. Exemplary Norepinephrine Reuptake Inhibitors

There are a variety of norepinephrine (NE) reuptake inhibitors available which can be used in the method of the present invention. Suitable norepinephrine reuptake inhibitors of use in the present invention include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

In certain embodiments, the subject method utilizes reboxetine, a selective norepinephrine reuptake inhibitor. Reboxetine (Edronax™), 2-[α-(2-ethoxy) phenoxybenzyl]morpholine, may be administered as the racemate or as a purified or semi-purified stereoisomer. It was first taught by U.S. Pat. No. 4,229,449, which describes its utility for the treatment of depression. Reboxetine is a selective norepinephrine reuptake inhibitor. The term “reboxetine” will be used here to refer to any acid addition salt or the free base of the molecule existing as the racemate or either enantiomer.

Other examples of NE reuptake inhibitors which may be useful in the methods of the present invention include:

Duloxetine, N-methyl-3-(1-naphthalenyloxy)-3-(2-thienyl)propanamine, is usually administered as the hydrochloride salt and as the (+) enantiomer. It was first taught by U.S. Pat. No. 4,956,388, which shows its high potency. The word “duloxetine” will be used here to refer to any acid addition salt or the free base of the molecule;

Venlafaxine is known in the literature, and its method of synthesis and its activity as an inhibitor of serotonin and norepinephrine uptake are taught by U.S. Pat. No. 4,761,501. Venlafaxine is identified as compound A in that patent; and

Milnacipran (N,N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake.

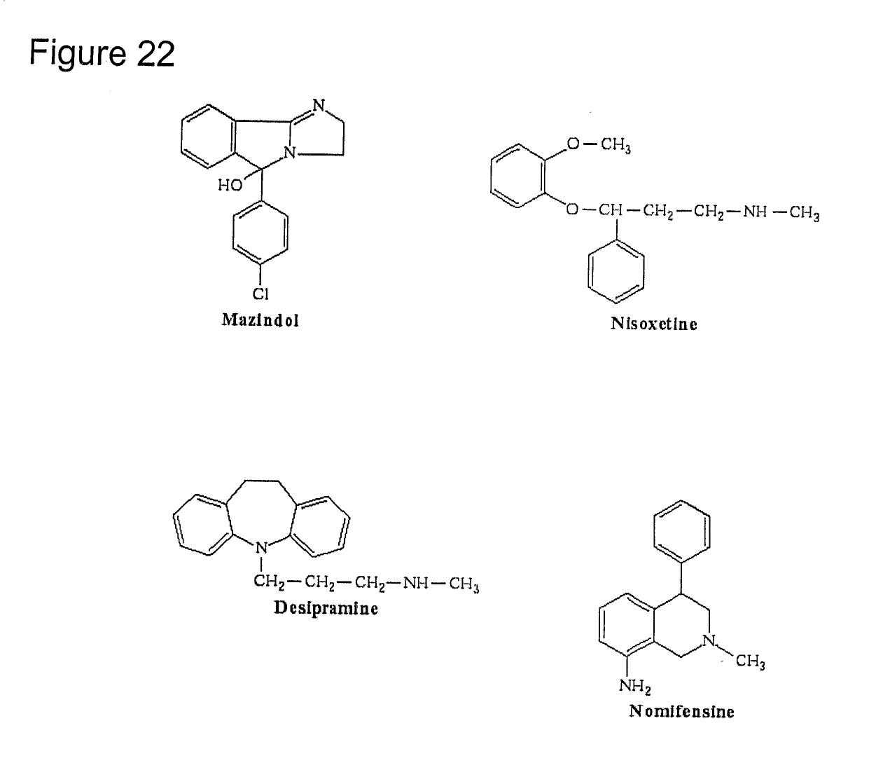

Still other examples of inhibitors of catecholamine uptake which may used in the subject method include Mazindol, Methylphenidate (Ritalin), Nefazodone (Serzone), Venlafaxine, Desipramine, Nisoxetine, Sibutramine and Nomifensine. See FIG. 22 for exemplary structures.

Another class of compounds include the benzodiazapene antidepressants of the norepinephrine reuptake inhibitor type.

In certain preferred embodiments, the subject reuptake inhibitors inhibit presynaptic norepinephrine reuptake with a Ki of 100 nM or less, more preferably 10 nM or less or even 1 nM or less. In certain embodiments, the subject reuptake inhibitors are selective and specific norepinephrine, e.g., relative to other catecholamines such as serotonin; e.g., the subject reuptake inhibitor has over 10 times greater selectivity for blocking norepinephrine as compared to dopamine and serotonin (5-HT) reuptake, and more preferably has more than 100 times greater selectivity compared to serotonin and more than 100 or even 1000 times greater selectivity compared to dopamine. In certain preferred embodiments, the subject reuptake inhibitors are at least 10 times more potent at blocking noradrenergic neurons as compared to serotonergic neurons.

The subject reuptake inhibitors can be provided in the form of pharmaceutical salts and as prodrugs.

The compounds of the present invention may also be provided in the form of prodrugs, e.g., to protect a drug from being altered while passing through a hostile environment, such as the digestive tract. Prodrugs can be prepared by forming covalent linkages between the drug and a modifier. See, for example, Balant at al., Eur. J. Drug Metab. Pharmacokinetics, 1990, 15(2), 143-153. The linkage is usually designed to be broken under defined circumstances, e.g., pH changes or exposure to specific enzymes. The covalent linkage of the drug to a modifier essentially creates a new molecule with new properties such as an altered log P value and/or as well as a new spatial configuration. The new molecule can have different solubility properties and be less susceptible to enzymatic digestion. For general references on prodrug design and preparation, see: Bundraard, Design of Prodrugs, Elsevier Science Pub. Co., N.Y. (1985), and Prodrugs as Novel Drug Delivery Systems Symposium, 168.sup.th Annual Meeting, American Chemical Society, Atlantic City, N.J., Eds. T. Higuchi and V. Stella, ACS Symposium Series 14, 1975, which are herein incorporated by reference.

B. Generation of Animal Models to Test Agents

Animal models for studying fornix-mediated memory consolidation have previously been described. See, for example, Taubenfield et al., Supra. The fornix-lesioned animals can be used for drug screening, e.g., to identify dosages of the subject reuptake inhibitors which enhance memory consolidation. The lesioned mammal can have a lesion of the fornix or a related brain structure that disrupts memory consolidation (e.g., perirhinal cortex, amygdala, medial septal nucleus, locus coeruleus, hippocampus, mammallary bodies). Lesions in the mammal can be produced by mechanical or chemical disruption. For example, the fornix lesion can be caused by surgical ablation, electrolytic, neurotoxic and other chemical ablation techniques, or reversible inactivation such as by injection of an anesthetic, e.g., tetrodotoxin or lidocaine, to temporarily arrest activity in the fornix.

To further illustrate, fimbrio-fornix (rodents) and fornix (primates) lesions can be created by stereotactic ablation. In particular, neurons of the fornix structure are axotomized, e.g., by transection or aspiration (suction) ablation. A complete transection of the fornix disrupts adrenergic, cholinergic and GABAergic function and electrical activity, and induces morphological reorganization in the hippocampal formation. In general, the fornix transection utilized in the subject method will not disconnect the parahippocampal region from the neocortex. In those embodiments, the fornix transection will not disrupt functions that can be carried out by the parahippocampal region independent of processing by the hippocampal formation, and hence would not be expected to produce the full-blown amnesia seen following more complete hippocampal system damage.

In one embodiment, the animal can be a rat. Briefly, the animals are anesthetized, e.g., with intraperitoneal injections of a ketamine-xylazine mixture and positioned in a Kopf stereotaxic instrument. A sagittal incision is made in the scalp and a craniotomy is performed extending 2.0 mm posterior and 3.0 mm lateral from Bregma. An aspirative device, e.g., with a 20 gauge tip, is mounted to a stereotaxic frame (Kopf Instruments) and fimbria-fornix is aspirated by placing the suction tip at the correct sterotaxic location in the animals brain. Unilateral aspirative lesions are made by suction through the cingulate cortex, completely transecting the fimbria fornix unilaterally, and (optionally) removing the dorsal tip of the hippocampus as well as the overlying cingulate cortex to inflict a partial denervation on the hippocampus target. See also, Gage et al., (1983) Brain Res. 268:27 and Gage et al. (1986) Neuroscience 19:241.

In another exemplary embodiment, the animal can be a monkey. The animal can be anesthetized, e.g., with isoflurane (1.5-2.0%). Following pretreatment with mannitol (0.25 g/kg, iv), unilateral transections of the left fornix can be performed, such as described by Kordower et al. (1990) J. Comp. Neurol., 298:443. Briefly, a surgical drill is used to create a parasagittal bone flap which exposes the frontal superior sagittal sinus. The dura is retracted and a self-retaining retractor is used to expose the interhemispheric fissure. The corpus callosum is longitudinally incised. At the level of the foramen of Monro, the fornix is easily visualized as a discrete 2-3 mm wide white fiber bundle. The fornix can be initially transected using a ball dissector. The cut ends of the fornix can then be suctioned to ensure completeness of the lesion.

In still other illustrative embodiments, the fornix lesion can be created by excitotoxically, or by other chemical means, inhibiting or ablating fornix neurons, or the cells of the hippocampus which are innervated by fornix neurons. In certain preferred embodiments, the fornix lesion is generated by selective disruption of particular neuronal types, such as fornix cholinergic and adrenergic neurons.

For instance, the afferant fornix signals to the hippocampus due to cholinergic neurons can be ablated by atropine blockade. Another means for ablation of the cholinergic neurons is the use of 192IgG-saporin (192IgG-sap), e.g., intraventricularly injection into the fornix and hippocampus. The agents such as 6-OHDA and ibotenic acid can be used to selectively destroy fornix dopamine neurons as part of the ablative regimen.

In preferred embodiments, the animal is a non-human mammal, such as a dog, cat, horse, cow, pig, sheep, goat, chicken, monkey, ape, rat, rabbit, etc. In certain preferred embodiments, the animal is a non-human primate. In other preferred embodiment, the animal is a rodent.

There are a variety of tests for cognitive function, especially learning and memory testing, which can be carried our using the lesioned and normal animals. Learning and/or memory tests include, for example, inhibitory avoidance, contextual fear conditioning, visual delay non-match to sample, spatial delay non-match to sample, visual discrimination, Barnes circular maze, Morris water maze, and radial arm maze tests.

An exemplary passive avoidance test utilizes an apparatus that consists of a lit chamber that can be separated from a dark chamber by a sliding door. At training, the animal is placed in the lit chamber for some period of time, and the door is opened. The animal moves to the dark chamber after a short delay—the latency—that is recorded. Upon entry into the dark chamber, the door is shut closed and a foot shock is delivered. Retention of the experience is determined after various time intervals, e.g., 24 or 48 hours, by repeating the test and recording the latency. The protocol is one of many variants of the passive avoidance procedures (for review, see Rush (1988) Behav Neural Biol 50:255).

An exemplary maze testing embodiment is the water maze working memory test. In general, the method utilizes an apparatus which consists of a circular water tank. The water in the tank is made cloudy by the addition of milk powder. A clear plexiglass platform, supported by a movable stand rest on the bottom of the tank, is submerged just below the water surface. Normally, a swimming rat cannot perceive the location of the platform but it may recall it from a previous experience and training, unless it suffers from some memory impairment. The time taken to locate the platform is measured and referred to as the latency. During the experiment, all orientational cues such as ceiling lights, etc., remain unchanged. Longer latencies are generally observed with rats with some impairment to their memory.

Another memory test includes the eyeblink conditioning test, which involves the administration of white noise or steady tone that precedes a mild air puff which stimulates the subject's eyeblink.

Still another memory test which can be used is fear conditioning, e.g., either “cued” and “contextual” fear conditioning. In one embodiment, a freeze monitor administers a sequence of stimuli (sounds, shock) and then records a series of latencies measuring the recovery from shock induced freezing of the animal.

Another memory test for the lesioned animals is a holeboard test, which utilizes a rotating holeboard apparatus containing (four) open holes arranged in a 4-corner configuration in the floor of the test enclosure. A mouse is trained to poke its head into a hole and retrieve a food reward from a “baited” hole which contains a reward on every trial. There is a food reward (e.g., a Froot Loop) in every exposed hole which is made inaccessible by being placed under a screen. The screen allows the odor of the reward to emanate from the hole, but does not allow access to the reinforcer. When an individual hole is baited, a reward is placed on top of the screen, where it is accessible. The entire apparatus rests on a turntable so that it may be rotated easily to eliminate reliance on proximal (e.g., olfactory) cues. A start tube is placed in the center of the apparatus. The subject is released from the tube and allowed to explore for the baited (“correct”) hole.

As set out above, one use for the fornix-lesioned animals is for testing reuptake inhibitors for ability to enhance or inhibit memory consolidation, as well as for side effects and toxicity. In general, the subject method utilizes an animal which has been manipulated to create at least partial disruption of fornix-mediated signalling to the hippocampus, the disruption affecting memory consolidation and learned behavior in the animal. The animal is conditioned with a learning or memory regimen which results in learned behavior in the mammal in the absence of the fornix lesion. Reuptake inhibitors are administered to the animal in order to assess their effects on memory consolidation. An increase in learned behavior, relative to the absence of the test agents, indicates that the administered combination enhances memory consolidation.

In the methods of the present invention, retention of the learned behavior can be determined, for example, after at least about 12-24 hours, 14-22 hours, 16-20 hours and or 18-19 hours after completion of the learning phase to determine whether the agents promote memory consolidation. In a particular embodiment, retention of the learned behavior can be determined 24 hours after completion of the learning phase.

As used herein, a “control mammal” can be an untreated lesion mammal (i.e., a lesion animal receiving no agents or not the same combinations to be assessed), a trained control mammal (i.e., a mammal that undergoes training to demonstrate a learned behavior without any lesion) and/or an untrained control mammal (i.e., a mammal with or without a lesion, that receives no training to demonstrate a learned behavior).

C. Pharmaceutical Preparations of Reuptake Inhibitors

In another aspect, the present invention provides pharmaceutical preparations comprising the subject reuptake inhibitors. The reuptake inhibitors for use in the subject method may be conveniently formulated for administration with a biologically acceptable, non-pyrogenic, and/or sterile medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the reuptake inhibitors, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Pharmaceutical formulations of the present invention can also include veterinary compositions, e.g., pharmaceutical preparations of the reuptake inhibitors suitable for veterinary uses, e.g., for the treatment of live stock or domestic animals, e.g., dogs.

Methods of introduction may also be provided by rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, can be used to form an implant for the sustained release of a reuptake inhibitor at a particular target site.

The preparations of the present invention may be given orally, parenterally, topically, or rectally. They are of course given by forms suitable for each administration route. For example, they are administered in tablets or capsule form, by injection, inhalation, eye lotion, ointment, suppository, controlled release patch, etc. administration by injection, infusion or inhalation; topical by lotion or ointment; and rectal by suppositories. Oral and topical administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular reuptake inhibitors employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with other antimicrobial agents such as penicillins, cephalosporins, aminoglycosides and glycopeptides. Conjunctive therapy thus includes sequential, simultaneous and separate administration of the active compound in a way that the therapeutic effects of the first administered one is not entirely disappeared when the subsequent is administered.

While it is possible for a compound of the present invention to be administered alone, it is preferable to administer the compound as a pharmaceutical formulation (composition). The reuptake inhibitors according to the invention may be formulated for administration in any convenient way for use in human or veterinary medicine.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water.

The phrase “pharmaceutically acceptable carrier” as used herein means a pharmaceutically acceptable material, composition or vehicle, such as a liquid or solid filter, diluent, excipient, solvent or encapsulating material, involved in carrying or transporting the subject regulators from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present reuptake inhibitors may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active reuptake inhibitor.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the reuptake inhibitors in the proper medium. Absorption enhancers can also be used to increase the flux of the reuptake inhibitors across the skin. The rate of such flux can be controlled by either providing a rate-controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and Co., San Francisco, U.S.A., 1969 or “Livestock Feeds and Feeding” and B books, Corvallis, Ore., U.S.A., 1977).

IV. Exemplary Uses of the Compounds of the Invention.

In various embodiments, the present invention contemplates modes of treatment and prophylaxis which utilize one or more of the subject catecholamine reuptake inhibitors. These agents may be useful for increasing the occurrence of memory consolidation (LTP) or decreasing or preventing the effects of defects in an animal which mitigate memory consolidation. In other embodiments, the preparations of the present invention can be used simply to enhance normal memory function.

In certain embodiments, the subject method can be used to treat patients who have been diagnosed as having or at risk of developing disorders in which diminished declarative memory is a symptom, e.g., as opposed to procedural memory. The subject method can also be used to treat normal individuals for whom improved declarative memory is desired.

Memory disorders which can be treated according to the present invention may have a number of origins: a functional mechanism (anxiety, depression), physiological aging (age-associated memory impairment, mild cognitive impairment, etc.), drugs, or anatomical lesions (dementia). Indications for which such preparations may be useful include learning disabilities, memory impairment, e.g., due to toxicant exposure, brain injury, brain aneurysm, age, schizophrenia, epilepsy, mental retardation in children, and senile dementia, including Alzheimer's disease.

Although in certain embodiments, attention deficit disorder (ADD), attention deficit hyperactivity disorder (ADHD), and AIDS-related dementia may respond to treatment with a subject compound, in certain embodiments, the patient's memory loss is not associated with one of these conditions.

An attention-deficit disorder (ADD) is a developmental disorder characterized by developmentally inappropriate degrees of inattention, overactivity, and impulsivity. Symptoms are neurologically based, arise in early childhood, and are chronic in nature in most cases. Symptoms are not due to gross neurological impairment, sensory impairment, language or motor impairment, mental retardation, or emotional disturbance.

ADD with and without hyperactivity are separate and unique childhood disorders. They are not subtypes of an identical attention disturbance. It has been noted that children with ADD/−H are more frequently described as depressed, learning disabled, or “lazy” while children with ADD/+H are more frequently labeled as conduct or behavior disordered.

Characteristics of ADHD have been demonstrated to arise in early childhood for most individuals. This disorder is marked by chronic behaviors lasting at least six months with an onset often before seven years of age. At this time, four subtypes of ADHD have been defined. These include the following:

1. ADHD—Inattentive type

2. ADHD—hyperactive/impulsive type

3. ADHD—combined type

4. ADHD—not otherwise specified is defined by an individual who demonstrates some characteristics but an insufficient number of symptoms to reach a full diagnosis. These symptoms, however, disrupt everyday life.

The American Psychiatric Association Diagnostic and Statistical Manual (DSM-IV) criteria for diagnosing ADHD include:

A. Either (1) or (2)

(1). six (or more) of the following symptoms of inattention have persisted for at least 6 months to a degree that is maladaptive and inconsistent with developmental level:

Inattention

(a) often fails to give close attention to details or makes careless mistakes in schoolwork, work, or other activities

(b) often has difficulty sustaining attention in tasks or play activities

(c) often does not seem to listen when spoken to directly

(d) often does not follow through on instructions and fails to finish schoolwork, chores, or duties in the workplace (not due to oppositional behavior or failure to understand instructions)

(e) often has difficulty organizing tasks and activities

(f) often avoids, dislikes, or is reluctant to engage in tasks that require sustained mental effort (such as schoolwork or homework).

(g) often loses things necessary for tasks or activities (e.g. toys, school assignments, pencils, books, or tools)

(h) is often easily distracted by extraneous stimuli

(i) is often forgetful in daily activities

(2). six (or more) of the following symptoms of hyperactivity-impulsivity have persisted for at least 6 months to a degree that is maladaptive and inconsistent with developmental level

Hyperactivity

(a) often fidgets with hands or feet or squirms in seat

(b) often leaves seat in classroom or in other situations in which remaining seated is expected

(c) often runs about or climbs excessively in situations in which it is inappropriate (in adolescents or adults, may be limited to subjective feelings of restlessness)

(d) often has difficulty playing or engaging in leisure activities quietly

(e) is often “on the go” or often acts as if “driven by a motor”

(f) often talks excessively

Impulsivity

(g) often blurts out answers before questions have been completed

(h) often has difficulty awaiting turn

(i) often interrupts or intrudes on others (e.g. butts into conversations or games)

B. Some hyperactive-impulsive or inattentive symptoms that caused impairment were present before age 7 years.

C. Some impairment from the symptoms is present in two or more settings (e.g. at school [or work] and at home).

D. There must be clear evidence of clinically significant impairment in social, academic, or occupational functioning. E. The symptoms do not occur exclusively during the course of a Pervasive Developmental Disorder, Schizophrenia, or other Psychotic Disorder and are not better accounted for by another mental disorder (e.g., Mood Disorder, Anxiety Disorder, Dissociative Disorder, or a Personality Disorder).

One aspect of the present invention relates to the combination of a catecholamine reuptake inhibitor and a dopamine reuptake inhibitor. A variety of dopamine transporter inhibitors (also called dopamine uptake inhibitors; herein referred to as active compounds) of diverse structure are known. See, e.g., S. Berger, U.S. Pat. No. 5,217,987; J. Boja et al., Molec. Pharmacol. 47, 779-786 (1995); C. Xu et al., Biochem. Pharmacol. 49, 339-50 (1995); B. Madras et al., Eur. J. Pharmacol. 267, 167-73 (1994); F. Carroll et al., J. Med. Chem. 37, 2865-73 (1994); A. Eshleman et al., Molec. Pharmacol. 45, 312-16 (1994); R. Heikkila and L. Manzino, Eur. J. Pharmacol. 103, 241-8 (1984). Dopamine transporter inhibitors are, in general, ligands that bind in a stereospecific manner to the dopamine transporter protein. Examples of such compounds are:

(1) tricyclic antidepressants such as buprion, nomifensine, and amineptin;

(2) 1,4-disubstituted piperazines, or piperazine analogs, such as 1-[2-[bis(4-fluorophenyl)methoxy]ethyl]-4-(3-phenylpropyl)piperazine dihydrochloride (or GBR 12909), 1-[2-[bis(phenyl) methoxy]ethyl]-4-(3-phenylpropyl)piperazine dihydrochloride (for GBR12934), and GBR13069;

(3) tropane analogs, or (disubstituted phenyl) tropane-2 beta-carboxylic acid methyl esters, such as 3 [beta]-(4-fluorophenyl)tropane-2 [beta] carboxylic acid methyl ester (or WIN 35,428) and 3 [beta]-(4-iodophenyl)tropane-2[beta]-carboxylic acid isopropyl ester (RTI-121);

(4) substituted piperidines, or piperidine analogs, such as N-[1-(2-benzo[b]thiophenyl)cyclohexyl]piperidine, indatraline, and 4-[2-[bis(4-fluorophenyl)methoxy]ethyl]-1-(3-phenylpropyl)piperidine (or 0-526);

(5) quinoxaline derivatives, or quinoxaline analogs, such as 7-trifluoromethyl-4-(4-methyl-1-piperazinyl)pyrrolo[1,2-[alpha]]-quinoxaline (or CGS 12066b); and

(6) other compounds that are inhibitors of dopamine reuptake, such as mazindol, benztropine, bupropion, phencyclidine, methylphenidate, etc.

Accordingly, certain embodiments of the invention relates to a method for treating ADHD (adult or child), comprising co-administering (e.g., simultaneously or at different times) to the patient (human or other animal) an amount of a catecholaminc reuptake inhibitor sufficient to treat the attention component of ADHD, and an amount of a dopamine reuptake inhibitor sufficient to treat the movement disorder component. In certain embodiments, the catecholaminc reuptake inhibitor and the dopamine reuptake inhibitor are administered simultaneously. In certain embodiments, the catecholamine reuptake inhibitor and the dopamine reuptake inhibitor are administered as part of a single composition. In certain embodiments, the single composition is for oral administration or for transdermal administration.

In yet another aspect, the invention relates to a method for preparing a pharmaceutical preparation, comprising combining a catecholamine reuptake inhibitor, dopamine reuptake inhibitor, and a pharmaceutically acceptable excipient in a composition for simultaneous administration of the two drugs.

In still another aspect, the invention relates to a method for conducting a pharmaceutical business, by manufacturing a preparation of a catecholamine reputake inhibitor (or prodrug or metabolite thereof) and a dopamine reuptake inhibitor or a kit including separate formulations of each, and marketing to healthcare providers the benefits of using the preparation or kit in the treatment of ADHD.

In yet another aspect, the invention provides a method for conducting a pharmaceutical business, by providing a distribution network for selling the combinatorial preparations and kits, and providing instruction material to patients or physicians for using such preparation to treat ADHD.

In still a further aspect, the invention relates to a method for conducting a pharmaceutical business, by determining an appropriate formulation and dosage of a catecholamine reuptake inhibitor (or a prpdrug or metabolite thereof), a dopamine reuptake inhibitor to be co-administered in the treatment of ADHD, conducting therapeutic profiling of identified formulations for efficacy and toxicity in animals, and providing a distribution network for selling a preparation as having an acceptable therapeutic profile. In certain embodiments, the method further includes an additional step of providing a sales group for marketing the preparation to healthcare providers.

In yet another aspect, the invention provides a method for conducting a pharmaceutical business by determining an appropriate formulation and dosage of a catecholamine reuptake inhibitor, a dopamine reuptake inhibitor to be co-administered in the treatment of ADHD, and licensing, to a third party, the rights for further development and sale of the formulation.

In certain embodiments, the invention contemplates the treatment of amnesia. Amnesias are described as specific defects in declarative memory. Faithful encoding of memory requires a registration, rehearsal, and retention of information. The first two elements appear to involve the hippocampus and medial temporal lobe structures. The retention or storage appears to involve the heteromodal association areas. Amnesia can be experienced as a loss of stored memory or an inability to form new memories. The loss of stored memories is known as retrograde amnesia. The inability to form new memories is known as anterograde amnesia.

Complaints of memory problems are common. Poor concentration, poor arousal and poor attention all may disrupt the memory process to a degree. The subjective complaint of memory problems therefore must be distinguished from true amnesias. This is usually done at the bedside in a more gross evaluation and through specific neuropsychological tests. Defects in visual and verbal memory can be separated through such tests. In amnesias there is by definition a preservation of other mental capacities such as logic. The neurobiologic theory of memory described above would predict that amnesias would have relatively few pathobiologic variations. Clinically the problem of amnesias often appears as a result of a sudden illness in an otherwise healthy person.

Exemplary forms of amnesias which may be treated by the subject method include amnesias of short duration, alcoholic blackouts, Wemicke-Korsakoff's (early), partial complex seizures, transient global amnesia, those which are related to medication, such as triazolam (Halcion), and basilar artery migraines. The subject method may also be used to treat amnesias of longer duration, such as post concussive or as the result of Herpes simplex encephalitis.

In certain embodiments, this invention contemplates the treatment of the Anterior Communicating Artery Syndrome. This syndrome is prevalent among survivors of Anterior Communicating artery aneurysms and often includes anterograde amnesia, a specific deficit in new memory formation, with relative sparing of older memories as well as intelligence and attention. The Anterior Communicating Artery Syndrome may also include some personality changes and confabulation. There is a considerable anatomic and clinical evidence that the Anterior Communicating Artery Syndrome in man is a result of a focal lesion in the basal forebrain area (particularly the medial septal area), secondary to combined damage from the aneurysm and the surgical or endovascular treatment of the aneurysm.

In addition, the compounds of the invention may be useful in enhancing memory in normal individuals.

Exemplification

The invention now being generally described, it will be more readily understood by reference to the following examples which are included merely for purposes of illustration of certain aspects and embodiments of the present invention, and are not intended to limit the invention.

Background Information and Objective

The Inhibitory Avoidance (IA) task is a well-studied behavioral paradigm which can provide the researcher with a consistent and long-lasting measure of memory. The paradigm consists of one training trial and one retention trial. Test articles may be administered to the rats either before or after training. Improved memory, as a result of test compound administration, is evident as increased latency on the retention trial. The objective of the following experiments was to investigate the effects of methylphenidate on IA memory in the rat.

Experiment 1: Dose Response Testing (Methylphenidate)

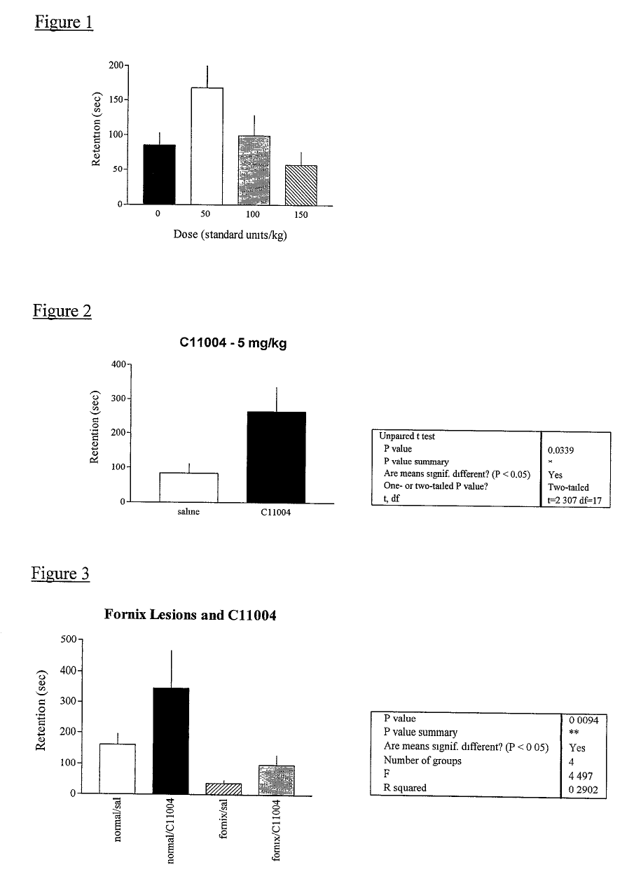

In this experiment, rats were injected with three different doses of methylphenidate (C11004) thirty minutes prior to training on the IA task. As can be seen from FIG. 1, a dose of 50 standard units/kg (10 standard units=1 mg) improved retention, while doses of 100 and 150 standard units/kg had no effect.

In order to verify this result, a second experiment was conducted. Rats were injected with 50 standard units/kg of methylphenidate and trained on the IA task. As can be seen from FIG. 2, this dose of methylphenidate significantly improved retention of the task. An unpaired t-test demonstrated that this enhancement was statistically significant (p<0.03).

Experiment 2: Time Course of Effectiveness (Methylphenidate)

In this experiment, the time of drug administration was varied in order to determine the optimal pre-training drug administration time. This experiment demonstrated that methylphenidate (50 standard units/kg) is most effective when administered to the rats one hour prior to training.

Experiment 3: Long-Term Retention (Methylphenidate)

This experiment was conducted in order to determine whether the enhanced retention observed in Experiment 2 was long-lasting. Rats received a second retention test one week after the first retention test. No additional training or drug was administered to the animals in the interim period.

Experiment 4: Effects on Lesioned Rats (Methylphenidate)

The findings of the above experiments are important, as they identify the most effective dose and time of administration for this compound. Moreover, the results demonstrate that methylphenidate improves memory in normal rats, and that this improvement is long lasting. In the next experiment, we investigated whether the performance of amnesic rats could be improved by administration of methylphenidate. In this experiment, control rats and rats with lesions of the fornix received injections of either saline or methylphenidate (5 mg/kg), and one hour later, were tested on the IA task.

As FIG. 3 illustrates, methylphenidate dramatically enhanced the performance of normal rats and in addition, appeared to improve the performance of the fornix lesion rats. A one-way ANOVA demonstrated that there was a significant difference between the performance of the four groups (F(3,36)=4.497,p<0.009). Student-Newman-Keuls post hoc tests revealed firstly that the performance of normal rats that received methylphenidate was significantly enhanced relative to all other experimental groups (p<0.05). In addition, the performance of fornix animals that received methylphenidate was not significantly different from normal, saline injected animals. These results demonstrate that methylphenidate is capable of enhancing memory in normal rats and has beneficial effects in brain damaged, amnesic rats.

Experiment 5: Dose Response Testing (Amphetamine)

In this experiment, rats were injected with three different doses of S-(+) amphetamine thirty minutes prior to being trained on the IA task. As can be seen from FIG. 4, a dose of 200 standard units of amphetamine improved retention of the task, while doses of 25, 50 and 100 standard units/kg had no effect.

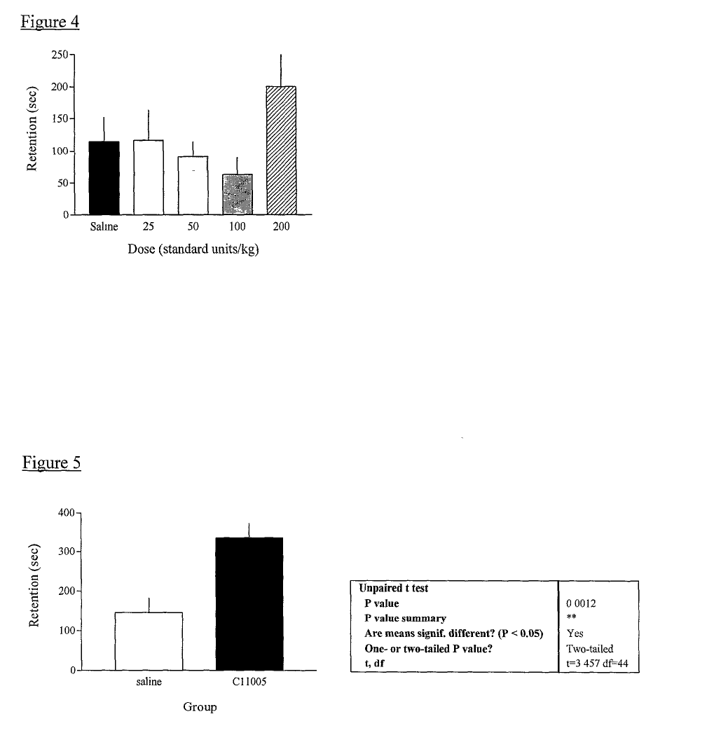

In order to verify this result, a second experiment was conducted. Rats were injected with 200 standard units/kg of amphetamine and trained on the IA task. As can be seen from FIG. 5, this dose of S-(+)amphetamine significantly improved retention of the task. An unpaired t-test demonstrated that this enhancement was statistically significant (p<0.01).

Experiment 6: Time Course of Effectiveness (Amphetamine)

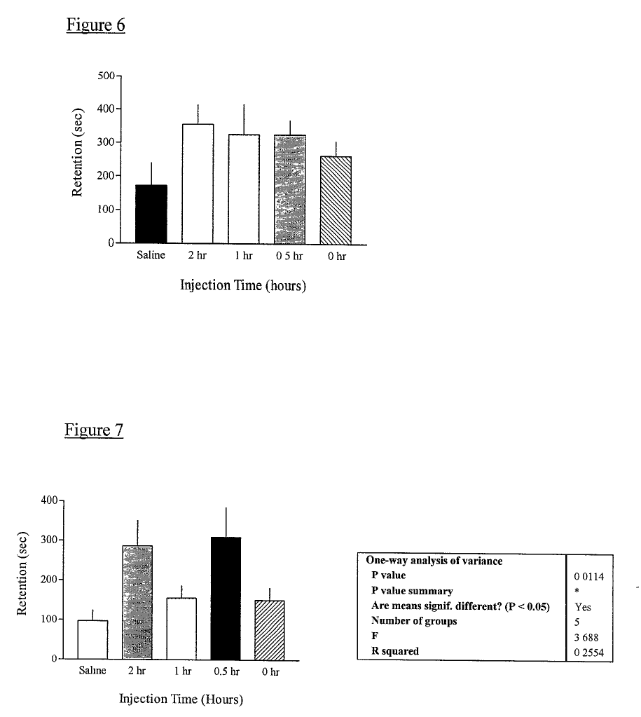

In this experiment, the time of drug administration was varied in order to determine the optimal pre-training drug administration time. FIG. 6 shows that S-(+) amphetamine (200 standard units/kg) is effective when administered to the rats between 0 and 2 hours prior to training.

Experiment 7: Long Term Retention (Amphetamine)

This experiment was conducted in order to determine whether the enhanced retention observed in Experiment 2 was long-lasting. Rats received a second retention test one week after the first retention test. No additional training or drug was administered to the animals in the interim period. FIG. 7 illustrates that rats that had received S-(+)amphetamine the previous week performed significantly better than rats that had received control injections of vehicle solution (F(4,47)=3.688, p<0.01).

Experiment 8: Effects on Lesioned Animals (Amphetamine)

The findings of the above experiments are important, as they identify the most effective dose and time of administration for this compound. Moreover, the results demonstrate that S-(+)amphetamine improves memory in normal rats, and that this improvement is long-lasting. In the next experiment, we investigated whether the performance of amnesic rats could be improved by administration of amphetamine. In this experiment, control rats and rats with lesions of the fornix received injections of either saline or amphetamine (200 standard units/kg), and one hour later, were tested on the IA task.

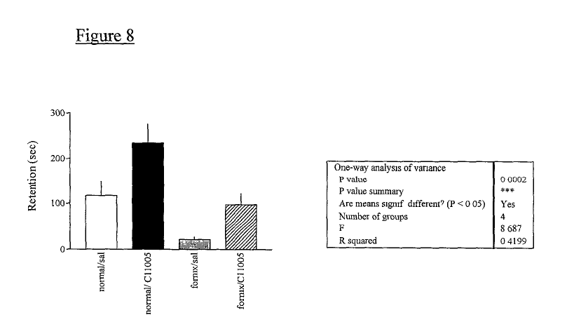

As FIG. 8 illustrates, S-(+)amphetamine dramatically enhanced the performance of normal rats and in addition, appeared to improve the performance of the fornix lesion rats. A one way ANOVA demonstrated that there was a significant difference between the performance of the four groups (F(3,36)=8.687, p<0.002). Student-Newman-Keuls post hoc tests revealed firstly that the performance of normal rats that received amphetamine was significantly enhanced relative to all other experimental groups (p<0.05). In addition, the performance of fornix animals that received amphetamine was not significantly different from normal, saline injected animals. These results demonstrate that amphetamine is capable of enhancing memory in normal rats and has beneficial effects in brain damaged, amnesic rats.

Experiment 9: Effects of Enantiomerically Enriched Amphetamine

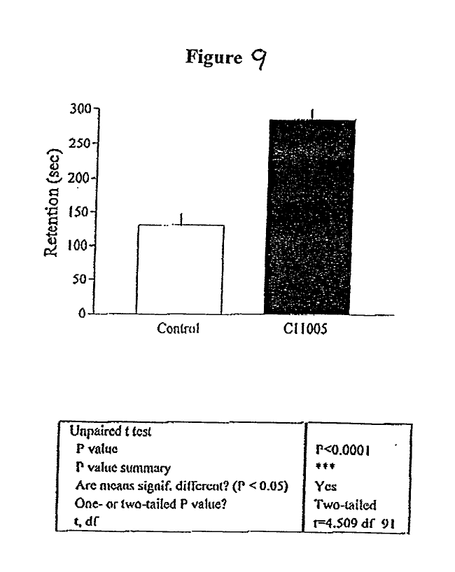

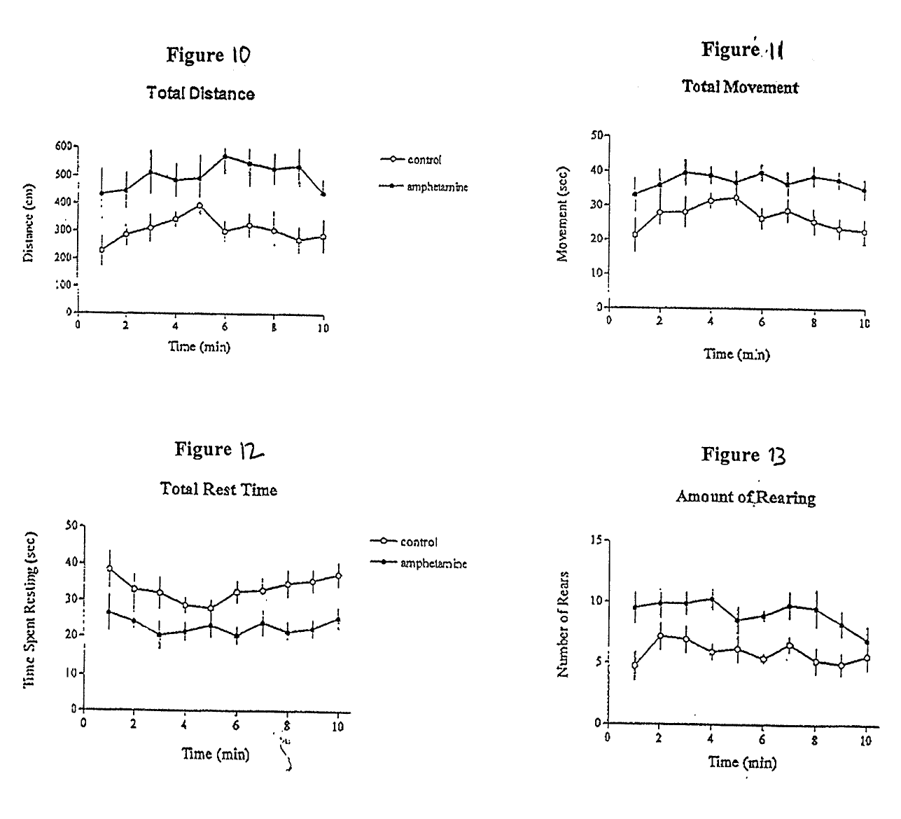

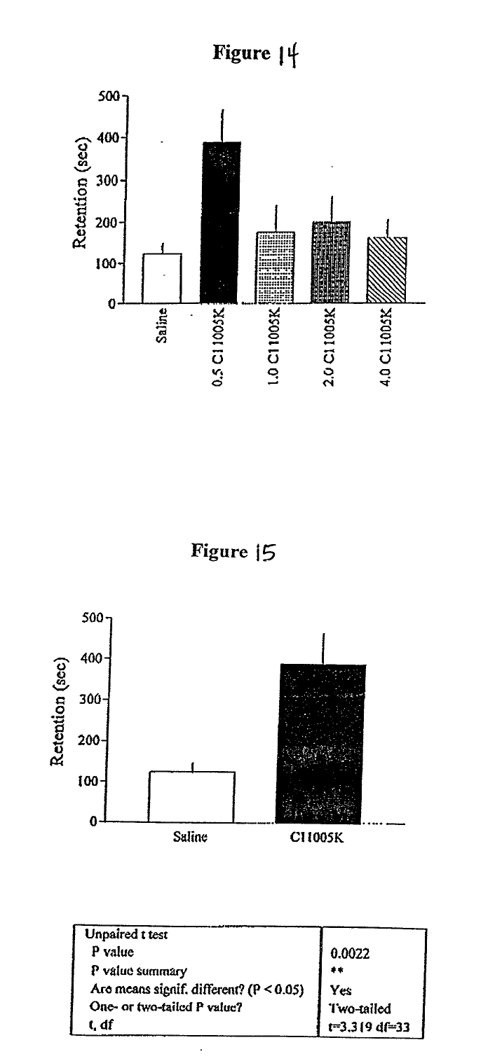

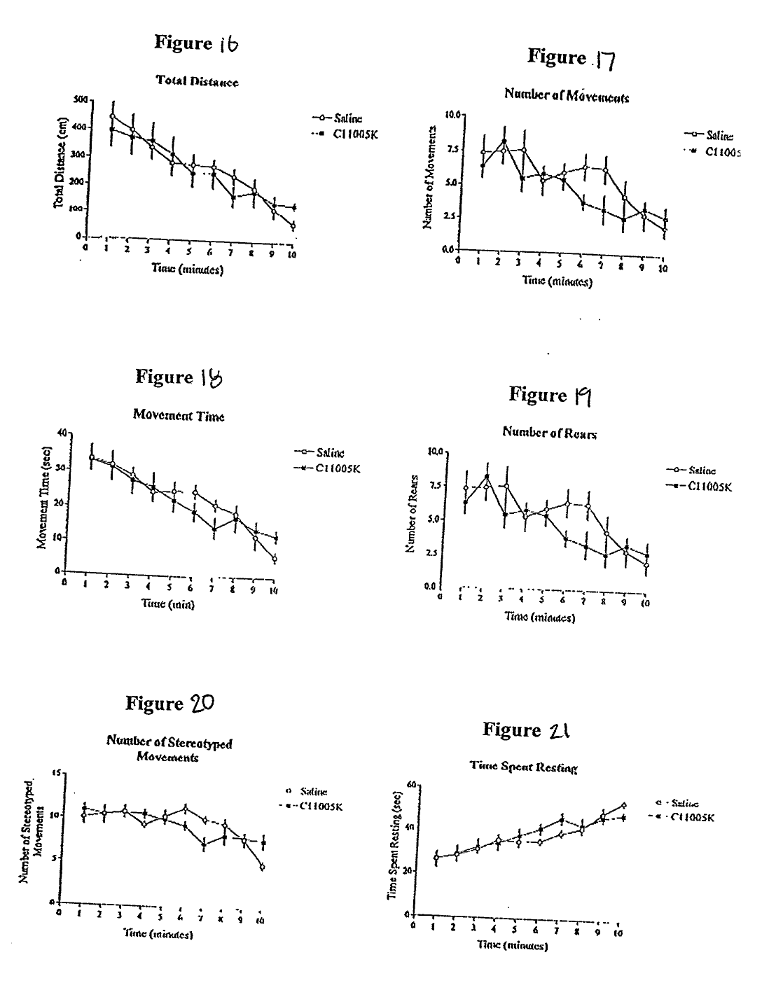

The effects of R-(−) vs. S-(+) amphetamine enantiomers on stimulation of memory consolidation and motor stimulation were compared. The S-(+) enantiomer of amphetamine is referred to as C11005 or amphetamine in the figures (FIGS. 9-13) and the R-(−) enantiomer of amphetamine is referred to as C11005K in the figures (FIGS. 15-21).

Effects of S-(+)Amphetamine on Inhibitory Avoidance

In this experiment, 48 rats were injected with 2 mg/kg (200 standard units) of S(+)amphetamine one hour prior to being trained on the IA task and were compared to a control group of rats injected with saline. The experiments were conducted with a 24 hour retention interval and a 0.46 mA shock intensity. As can be seen from FIG. 9, the dose of 200 standard units of S-amphetamine (C11005) improved retention of the task.

Effects of S-(+)Amphetamine on Activity Levels

In this experiment, 8 rats were injected with 2 mg/kg (200 standard units) of S(+)amphetamine and compared to a control group of rats injected with saline. Rat activity was monitored for a 10 minute period one hour after S-(+)amphetamine injection. As can be seen in FIGS. 10-13, treatment with S-(+)amphetamine had a significant effect on the activity levels of the rats as compared to the control group. Rats injected with S-(+)amphetamine had higher levels of activity on all of the activity tests for the entire 10 minute session.

Dose Response Testing of R-(−)Amphetamine (C11005K)

In this experiment, four groups of 10 rats were injected with different doses (0.5 mg/kg, 1.0 mg/kg, 2.0 mg/kg or 4.0 mg/kg) of the R-(−) enantiomer of amphetamine one hour prior to being trained on the IA task. The experiments were conducted with a 24 hour retention interval and a 0.46 mA shock intensity. As can be seen in FIG. 14, a much lower dose of R-(−)amphetamine is required for the same improved retention effect as obtained with S-(+)amphetamine (compare to FIG. 10). Increasing the dose above 0.5 kg/mg did not further improve the retention results obtained with this dose possibly indicating a saturation effect.

Effects of R-(−)Amphetamine (C11005K) on Inhibitory Avoidance

In order to verify the results from the dose response test, a second experiment with R-(−)amphetamine was conducted. Eighteen rats were injected with a dose of 0.5 mg/kg of R-(−)amphetamine one hour prior to being trained on the IA task. The R-(−) amphetamine treated rats were compared to control rats injected with saline. The experiments were conducted with a 24 hour retention interval and a 0.46 mA shock intensity. As can be seen in FIG. 15, this dose of R-(−)amphetamine significantly improved retention of the task. An unpaired t-test demonstrated that this enhancement was statistically significant (p<0.002).

Effects of R-(−)Amphetamine (C11005K) on Activity Levels

In this experiment, 8 rats were injected with 0.5 mg/kg of R-(−)amphetamine and compared to a control group of rats injected with saline. Rat activity was monitored for a 10 minute period one hour after R-(−)amphetamine injection. As can be seen in FIGS. 16-21, treatment with R-(−)amphetamine had no significant effects on the activity levels of the rats as compared to the control group. This data indicates that R-(−)amphetamine can provide improved memory consolidation without producing the motor stimulatory effects observed in the S-(+)amphetamine treated rats (compare to FIGS. 10-13).

### Equivalents

Those skilled in the art will recognize, or be able to ascertain using no more than routine experimentation, many equivalents to the specific embodiments of the invention described herein. Such equivalents are intended to be encompassed by the following claims.

All patents, publications, and other references cited above are hereby incorporated by reference in their entirety.

We claim:

1. A method for enhancing long term memory in an animal, comprising administering to the animal a formulation of a catecholamine reuptake inhibitor in an amount sufficient to enhance long-term memory in the animal.

2. The method of claim 1, wherein the catecholamine reuptake inhibitor is a norepinephrine reuptake inhibitor.

3. The method of claim 2, wherein the norepinephrine reuptake inhibitor is a tertiary amine tricyclics or a secondary amine tricyclics.

4. The method of claim 2, wherein the norepinephrine reuptake inhibitor is selected from the group consisting of amitriptyline, clomipramine, doxepin, imipramine, trimipramine, amoxapine, desipramine, maprotiline, nortriptyline, protriptyline, reboxetine, duloxetine, venlafaxine, milnacipran, mazindol, methylphenidate, nefazodone, nisoxetine, sibutramine and nomifensine.

5. The method of any of claims 2-3, wherein the norepinephrine reuptake inhibitor inhibit presynaptic norepinephrine reuptake with a Ki of 100 nM or less.

6. The method of any of claims 2-3, wherein the norepinephrine reuptake inhibitor has over 10 times greater selectivity for blocking norepinephrine reuptake as compared to inhibition of dopamine and serotonin (5-HT) reuptake.

7. The method of claim 5, wherein the norepinephrine reuptake inhibitor has over 10 times greater selectivity for blocking norepinephrine reuptake as compared to inhibition of dopamine and serotonin (5-HT) reuptake.

8. The method of any of claims 2-3, wherein the norepinephrine reuptake inhibitor is at least 10 times more potent at blocking noradrenergic neurons as compared to serotonergic neurons.

9. The method of claim 5, wherein the norepinephrine reuptake inhibitor is at least 10 times more potent at blocking noradrenergic neurons as compared to serotonergic neurons.

10. The method of claim 6, wherein the norepinephrine reuptake inhibitor is at least 10 times more potent at blocking noradrenergic neurons as compared to serotonergic neurons.

11. The method of claim 7, wherein the norepinephrine reuptake inhibitor is at least 10 times more potent at blocking noradrenergic neurons as compared to serotonergic neurons.

12. The method of claim 1, wherein the animal is further dosed with a neuronal growth factor, a neuronal survival factor, a neuronal tropic factor, a cholinergic activator, an adrenergic activator, a dopaminergic activator, a glutaminergic activator or an agent that stimulates the PKC or PKA pathways.

13. The method of claim 1, used for the prophylaxis or treatment of an animal susceptible to or suffering from anxiety, depression, age-associated memory impairment, minimal cognitive impairment, amnesia, dementia, learning disabilities, memory impairment associated with toxicant exposure, brain injury, stroke, schizophrenia, epilepsy, mental retardation, Alzheimer's disease, age, attention deficit disorder, attention deficit hyperactivity disorder, or AIDS-related dementia.

14. The method of claim 1, for treating and/or preventing memory impairment.

15. The method of claim 14, wherein the memory impairment results from one or more of anxiety, depression, age-associated memory impairment, minimal cognitive impairment, amnesia, dementia, learning disabilities, memory impairment associated with toxicant exposure, brain injury, brain aneurysm, Parkinson's disease, head trauma, Huntington's disease, Pick's disease, Creutzfeldt-Jakob disease, stroke, schizophrenia, epilepsy, mental retardation, Alzheimer's disease, age, attention deficit disorder, attention deficit hyperactivity disorder, or AIDS-related dementia.

16. The method of claim 1 for enhancing memory in normal individuals.

17. The method of claim 1, wherein said catecholamine reuptake inhibitor is provided in an amount sufficient to enhance long-term memory in a patient by a statistically significant amount when assessed by a standardized performance test.

18. The method of claim 1, wherein said catecholamine reuptake inhibitor is provided in an amount sufficient to enhance long-term memory in a patient by a statistically significant amount when assessed by one or more of a Cambridge Neuropsychological Test Automated Battery (CANTAB); a Children's Memory Scale (CMS); a Contextual Memory Test; a Continuous Recognition Memory Test (CMRT); a Denman Neuropsychology Memory Scale; a Fuld Object Memory Evaluation (FOME); a Graham-Kendall Memory for Designs Test; a Guild Memory Test; a Learning and Memory Battery (LAMB); a Memory Assessment Clinic Self-Rating Scale (MAC-S); a Memory Assessment Scales (MAS); a Randt Memory Test; a Recognition Memory Test (RMT); a Rivermead Behavioral Memory Test; a Russell's Version of the Wechsler Memory Scale (RWMS); a Test of Memory and Learning (TOMAL); a Vermont Memory Scale (VMS); a Wechsler Memory Scale; and a Wide Range Assessment of Memory and Learning (WRAML).

19. The method of claim 1, wherein said catecholamine reuptake inhibitor is provided in an amount sufficient to enhance long-term memory in a patient by a statistically significant amount when assessed by a Providence Recognition Memory Test.

20. The method of any of claims 1, 2 or 13, for veterinary treatment of a non-human mammal.

21. The method of any of claims 1, 2 or 13, for treatment of a human.

22. A medicament for enhancing memory in an animal, comprising a formulation of a catecholamine reuptake inhibitor in an amount sufficient to enhance long-term memory in the animal.

23. A method for preparing a formulation for enhancing memory consolidation in an animal, comprising preparing a pharmaceutical preparation comprising one or more catecholamine reuptake inhibitors in an amount sufficient to enhance long-term memory in the animal.

24 The medicament of claim 22, or the method of claim 23, wherein the catecholamine reuptake inhibitor is a norepinephrine reuptake inhibitor.

25. A kit comprising one or more catecholamine reuptake inhibitors, provided in single oral dosage form or as a transdermal patch, in an amount sufficient for enhancing memory in a patient, and in association with instructions (written and/or pictorial) describing the use of the kit for enhancing memory, and optionally, warnings of possible side effects and drug-drug or drug-food interactions.

26. A method for conducting a pharmaceutical business, comprising:
a. manufacturing a kit of claim 25 or medicament of claim 22; and
b. marketing to healthcare providers the benefits of using the kit or medicament to enhance memory of treated patients.

27. A method for conducting a pharmaceutical business, comprising:
a. providing a distribution network for selling a kit of claim 25 or medicament of claims 22; and
b. providing instruction material to patients or physicians for using the kit or medicament to enhance memory of treated patients.

28. A method for conducting a pharmaceutical business, comprising:
a. determining an appropriate dosage of a catecholamine reuptake inhibitor(s) to enhance memory function in a class of patients;
b. conducting therapeutic profiling of one or more formulations of the catecholamine reuptake inhibitor(s) identified in step (a), for efficacy and toxicity in animals; and
c. providing a distribution network for selling a the formulations identified in step (b) as having an acceptable therapeutic profile.

29. The method of claim 28, including an additional step of providing a sales group for marketing the preparation to healthcare providers.

30. A method for conducting a pharmaceutical business, comprising:
a. determining an appropriate dosage of a catecholamine reuptake inhibitor to enhance memory function in a class of patients; and
b. licensing, to a third party, the rights for further development and sale of the catecholamine reuptake inhibitor for enhancing memory.

31. The method of claim 30, wherein the class of patients suffer from memory impairment.

32. The method of claim 31, wherein the memory impairment results from one or more of anxiety, depression, age-associated memory impairment, minimal cognitive impairment, amnesia, dementia, learning disabilities, memory impairment associated with toxicant exposure, brain injury, brain aneurysm, Parkinson's disease, head trauma, Huntington's disease, Pick's disease, Creutzfeldt-Jakob disease, stroke, schizophrenia, epilepsy, mental retardation, Alzheimer's disease, age, attention deficit disorder, attention deficit hyperactivity disorder, or AIDS-related dementia.

33. The method of claim 30, wherein the class of patients are normal individuals.
